(19) United States
(12) Patent Application Publication
(10) Pub. No.: US 2002/0127189 A1
(43) Pub. Date: Sep. 12, 2002

(54) Chewing gum containing synephrine, ephedrine and caffeine

(76) Inventors: Thomas R. Myers, Redwood, CA (US); Alan Shugarman, Salt Lake City, UT (US); Jeffrey A. Felliciano, West Hollywood, CA (US); Luke R. Bucci, Reno, NV (US)

Correspondence Address: NIXON & VANDERHYE P.C., 8th Floor, 1100 North Glebe Road, Arlington, VA, 22201, US

(21) Appl. No.: 09/803,646

(22) Filed: Mar. 12, 2001

(51) Int. Cl. A61K 31/522; A61K 9/68

(52) U.S. Cl. 424/48; 514/263.32

(57) ABSTRACT

Chewing gum compositions comprising synephrine, ephedrine and caffeine are provided. The compositions are useful for supporting thermogenesis and anorectic effects, while generating enhanced mental alertness and improved energy levels.

The present invention relates to chewing gum compositions and their use for oral delivery of synephrine, ephedrine and caffeine to humans. The compositions are useful for supporting thermogenesis and anorectic activity, while generating enhanced mental alertness and improved energy levels.

### BACKGROUND OF THE INVENTION

The problem of obesity in humans and the effect excess weight on personal health is well known. Obesity results from over-eating, or the combination of over-eating with inactivity. More recently, obesity has been attributed, in certain cases, to a genetic predisposition in combination with poor diet and exercise habits. It has been suggested that predisposition to obesity is associated with a defect in the sympathetic nervous system. This defect is manifested as a high efficiency in food utilization and a reduced thermogenic response to food intake.

In normal persons, food intake results in a thermogenic response, i.e. an increase in body temperature in which the caloric content of food is expended as heat. Some studies suggest that persons with a genetic predisposition to obesity are metabolically less efficient than lean persons, storing excess caloric energy as body fat. In obese persons, thermogenic defects may make a significant contribution to weight gain in the absence of controlled food intake.

It is known that ephedrine stimulates thermogenesis in laboratory animals, possibly by stimulating brown adipose tissue. In theory, catecholamines activate thermogenesis in brown adipose tissue in animals by binding adrenergic receptors. Numerous studies have been published on the thermogenic response of humans and mammals to ephedrine treatment. The anorectic effect of ephedrine has also been investigated in rats. Ephedrine, used alone, results in increased thermogenesis, but also undesirable side effects in man, such as elevated blood pressure and tremors.

Studies have also been reported investigating combinations of ephedrine with additional compounds, such as caffeine. A composition containing ephedrine, caffeine and phenobarbitol has been reported as inducing loss of appetite and weight loss in humans. This composition, popularly known as the “Elsinore pill” was widely prescribed. However, serious side effects such as cutaneous reactions (tremors) were reported with this composition.

Ephedrine/caffeine compositions without the presence of phenobarbitol have also been investigated in attempts to reduce the side effects of the Elsinore pill. However, patients receiving this “modified Elsinore pill” continue to suffer from transient tremors similar to the effects seen with the Elsinore pill.

More recent studies of the thermogenic effects in humans of mixtures of ephedrine and methylxanthines, such as caffeine and theophylline, have been reported. These studies suggest that ephedrine/methylxanthine mixtures are more effective than ephedrine given alone. Methylxanthines are reported as potentiating the thermogenic anti-obesity effect of ephedrine leading to normalization of body weight and body composition. However, reports have also been published suggesting that caffeine has no potentiating effect on the action of ephedrine.

A need continues to exist for improved weight loss compositions which are safe, effective and exhibit reduced side effects in humans. The present invention seeks to satisfy that need.

### SUMMARY OF THE INVENTION

It has now been found surprisingly that chewing gum formulations of caffeine, synephrine and the additional presence of ephedrine provide an effective medium for oral administration of reduced dosages of caffeine, synephrine and ephedrine. The administration of these compounds via the oral route permits sufficiently high blood levels to be achieved with reduced incidence of adverse side-effects and with the added benefits of inducement of weight loss through thermogenesis and anorectic activity, while generating enhanced mental alertness and improved energy levels.

In a first aspect, the present invention provides a chewing gum composition comprising synephrine, ephedrine and caffeine.

In another aspect, there is provided a method of oral delivery of an effective amount of synephrine, ephedrine and caffeine to a human, comprising administering a chewing gum composition comprising synephrine, ephedrine and caffeine.

### DETAILED DESCRIPTION OF THE INVENTION

The composition of the invention comprises an effective amount of synephrine, ephedrine and caffeine. The composition is typically in the form of chewing gum sticks, balls, tabs, gumballs, or other gum form, but may be in other forms, such as mints, candies, chews, capsules, tablets and sachets.

Synephrine (Citrus aurantium—bitter orange) is a synthetic sympathomimetic drug which exhibits various types of pharmacological activity, such as vasoconstriction, blood pressure elevation and bronchial muscle relaxation. Synephrine is added typically as an herbal extract of Zhi Shi, Citrus Aurantium, Bitter Citrus, or Bitter Orange and also as synephrine HCl, and is present in the composition in an amount of about 0.5 mg to 100 mg per dose, more usually about 3 mg per dose.

The ingredient sources of the ephedrine alkaloids include raw botanicals and extracts from botanical sources such as: Ma huang, Ephedra, Chinese Ephedra, and epitonin. There are several names used for botanical products, primarily from Ephedra sinica Stapf, E. equistestina Bunge, E. intermedia var. tibetica Stapf and E. distachya L. (the Ephedras), that are sources of ephedrine alkaloids. The definition of ephedrine alkaloids includes: ephedrine, pseudoephedrine, norpseudoephedrine, norephedrine, methylephedrine, methylpseudoephedrine, and related alkaloids are naturally occurring chemical stimulants.

Ephedrine possesses two assymetric carbon atoms and is therefore available in four different stereoisomers, that is, (−)-ephedrine, (+)-ephedrine, (−)-.psi.-ephedrine and (+)-.psi.-ephedrine, as well as two racemic mixtures. The stereoisomer (−)-ephedrine is more effective and is therefore the preferred ephedrine stereoisomer for use in the present invention. However, the other stereoisomers of ephedrine are active in stimulating a thermogenic response and may be used in the present invention if desired. Herbal sources of ephedrine alkaloids are generally brown to black in color and consititute an extract to concentrate the “ephedrine alkaloids. Ephedrine is available as a white powder in technical and pharmaceutical grades. Typically, ephedrine and/or its alkaloids are present in an amount of about 0.5 mg to 100 mg per dose, more usually about 3-5 mg per dose.

The ephedrine used in the present invention may also be present as pharmaceutically acceptable salts if desired. Typical salts include hydrochloride, hydrobromide and maleate salts, although any pharmaceutically acceptable salt may be used.

The chewing gum composition of the present invention also includes caffeine. Caffeine is a natural chemical found in a variety of food products, such as coffee, tea, cocoa, chocolate, and various other beverages. Caffeine is known as an effective stimulant to increase energy and reduce drowsiness.

The caffeine is typically provided in the chewing gum composition as a raw material (3,7-Dihydro-1,3,7-trimethyl-1H-purine-2,6-dione). Caffeine salt compounds such as caffeine citrate, caffeine sodium benzoate, caffeine sodium salicylate, which may be more water soluble and less bitter than caffeine, may also be used in accordance with the present invention. There may also be sources of caffeine from plants such as Guarana, cocoa, coffee, or other plant extracted source of caffeine and its methylxanthine isomers (theophylline). The caffeine can be added to the chewing gum as a powder, as an aqueous dispersion, or dispersed in glycerin, propylene glycol, corn syrup, hydrogenated starch hydrolyzate, or any other compatible aqueous dispersion.

The caffeine is typically present in the chewing gum compositions in an amount of about 0.5-300 mg per dose, more usually about 40-60 mg per dose. Usually, the caffeine is present in an amount of about 0.5% to 5.0% by weight of the chewing gum composition, more usualy about 2.0% by weight. The chewing gum composition typically delivers about 20 to 100 mg of caffeine per stick of chewing gum, for example about 40-60 mg of caffeine per stick of chewing gum.

Optionally, the chewing gum compositions of the invention additionally comprise white willow, which has the effect of enhancing the effects of the ephedrine alkaloids, caffeine, and synephrine combination for thermogenesis and weight reduction. The white willow is typically present in an amount of about 0.5 mg to 3 grams, more usually about 50 mg to 2 grams.

Other substances which may be present are Schizandra chinensis, typically in an amount of about 0.5 mg to 2 grams, more usually about 25 mg to 1 gram, and iso-quescetin, typically in an amount of about 0.5 mg to 1 gram, more usually about 10 mg to 500 mg. Quercetin, a common plant flavanoid may also be present, typically in an amount of about 0.5 mg to 1 g, more usually about 45 mg.

A variety of different chewing gums may be utilized. For example, the chewing gums can be low or high moisture, sugar or sugarless, wax-containing or wax-free, low calorie (via high base or low calorie bulking agents), and/or may contain dental agents. Chewing gum generally consists of a water insoluble gum base, a water soluble portion, and flavors. The water soluble portion dissipates with a portion of the flavor of the gum over a period of time during chewing. The gum base portion is retained in the mouth throughout the chew. The insoluble gum base generally comprises elastomers, resins, fats and oils, softeners and inorganic fillers. The insoluble gum base can constitute about 5.0% to about 50%, by weight, of the chewing gum. The insoluble gum base generally comprises about 10% to about 30%, by weight, of the chewing gum.

The chewing gum base of the present invention generally contains about 15% to about 70% by weight synthetic elastomer, about 0% to about 35% by weight natural elastomer, about 5% to about 60% by weight elastomer plasticizer, about 5% to about 40% by weight filler, about 5% to about 40% by weight softener, and optional minor amounts (about 1% or less by weight) of miscellaneous ingredients such as colorants, antioxidants, etc. Synthetic elastomers may include, but are not limited to, polyisobutylene with GPC weight average molecular weight of about 10,000 to about 95,000, isobutylene-isoprene copolymer (butyl elastomer), styrene-butadiene, copolymers having styrene-butadiene ratios of about 1:3 to about 3:1, polyvinyl acetate having GPC weight average molecular weight of about 2,000 to about 90,000, polyisoprene, polyethylene, vinyl acetate-vinyl laurate copolymer having vinyl laurate content of about 5% to about 50% by weight of the copolymer, and combinations thereof.

Typically, the chewing gum composition includes additional sweeteners together with natural and artificial flavoring agents to reduce bitterness associated with the caffeine, ephedrine alkaloids, synephrine extracts. Possible sweeteners include, for example, acesulfame-K, aspartame, saccharin and xylitol. Natural and artificial flavors include, for example, monoammonium glycerrhizinate, glucono delta lactone, ethylmaltol, vanillin, creams, fruit flavors, and spices.

Fillers/texturizers may also be present. Examples include magnesium and calcium carbonate, ground limestone, silicate types such as magnesium and aluminum silicate, clay, alumina, talc, titanium oxide, mono-, di- and tri-calcium phosphate, cellulose polymers, such as wood, and combinations thereof. The filler may constitute between about 1% and about 60% by weight of the gum base. Preferably, the filler comprises about 5% to about 50% by weight of the gum base.

Softeners/emulsifiers may be present in order to optimize the chewability and mouth feel of the gum. The softeners, which are also known as plasticizers and plasticizing agents, generally constitute between approximately 0.5% to about 20% by weight of the chewing gum. Softeners may include glycerin, lecithin, and combinations thereof. Aqueous sweetener solutions such as those containing sorbitol, hydrogenated starch hydrolysates, corn syrup and combinations thereof, may also be used as softeners and binding agents in chewing gum. Other examples include tallow, hydrogenated tallow, hydrogenated and partially hydrogenated vegetable oils, cocoa butter, glycerol monostearate, glycerol triacetate, lecithin, mono-, di- and triglycerides, acetylated monoglycerides, fatty acids (e.g. stearic, palmitic, oleic and linoleic acids), and combinations thereof.

Colorants and whiteners are usually present. These may include FD&C-type dyes and lakes, fruit and vegetable extracts, titanium dioxide, and combinations thereof. In an embodiment, the composition includes Blue #1 Lake and Yellow #5 Lake as the colorants.

Sweeteners, flavoring agents, softeners, emulsifiers, colors, acidulants, fillers, antioxidants, and other components may be added as desired. Bulk sweeteners include both sugar and sugarless components. Bulk sweeteners typically constitute about 5% to about 95% by weight of the chewing gum, more typically, about 20% to about 80% by weight, and more commonly, about 30% to about 60% by weight of the gum. Sugar sweeteners generally include saccharide-containing components commonly known in the chewing gum art, including but not limited to, sucrose, dextrose, maltose, dextrin, dried invert sugar, fructose, levulose, glactose, corn syrup solids, and the like, alone or in combination. Sugarless sweeteners include, but are not limited to, sugar alcohols such as sorbitol, mannitol, xylitol, hydrogenated starch hydrolysates, maltitol, and the like, alone or in combination. High intensity artificial sweeteners can also be used, alone or in combination, with the above. Preferred sweeteners include, but are not limited to, sucralose, aspartame, salts of acesulfame, altitame, saccharin and its salts, cyclamic acid and its salts, glycerrhizinate, dihydrochalcones, thaumatin, monellin, and the like, alone or in combination. In order to provide longer lasting sweetness and flavor perception, it may be desirable to encapsulate or otherwise control the release of at least a portion of the artificial sweetener. Such techniques as wet granulation, wax granulation, spray drying, spray chilling, fluid bed coating, coacervation, and fiber extension may be used to achieve the desired release characteristics.

Combinations of sugar and/or sugarless sweeteners may be used in chewing gum. Additionally, the softener may also provide additional sweetness such as with aqueous sugar or alditol solutions. If a low calorie gum is desired, a low caloric bulking agent can be used. Examples of low caloric bulking agents include: polydextrose; Raftilose, Raftilin; Fructooligosaccharides (NutraFlora); Palatinose oligosaccharide; Guar Gum Hydrolysate (Sun Fiber); or indigestible dextrin (Fibersol). However, other low calorie bulking agents can be used.

A variety of flavoring agents can also be used, if desired. The flavor can be used in amounts of about 0.1 to about 20 weight percent of the gum, and preferably, about 0.1% to about 5% by weight. Flavoring agents may include essential oils, synthetic flavors or mixtures thereof including, but not limited to, oils derived from plants and fruits such as citrus oils, fruit essences, peppermint oil, spearmint oil, other mint oils, clove oil, oil of wintergreen, anise and the like. Artificial flavoring agents and components may also be used. Natural and artificial flavoring agents may be combined in any sensorially acceptable fashion.

By way of example, and not limitation, the chewing gums can have the following composition (all percentages by weight): about 50% to about 85% sugar; about 10% to about 30% gum base; about 5% to about 15% corn syrup; about 1% to about 10% natural and artificial flavors; about 1% to about 5% glycerine; about 0% to about 10% xylitol and about 1.5% to about 4.0% caffeine. In addition, in preferred embodiments, the composition further includes about 0% to about 1.5% glucono delta lactone; about 0% to about 0.5% aspartame; about 0% to about 0.6% acesulfame-K; about 0% to about 1.0% monoammonium glycerrhizinate (natural flavor); about 0% to about 0.05 Blue #1 Lake; and about 0% to about 0.05% Yellow #5 Lake (all percentages by weight).

Generally, the ingredients are mixed by first melting the gum base and adding it to the running mixer. The base may also be melted in the mixer itself. Color or emulsifiers may also be added at this time. A softener such as glycerin may also be added at this time, along with syrup and a portion of the bulking agent. Further portions of the bulking agent may then be added to the mixer. A flavoring agent is typically added with the final portion of the bulking agent. The ephedrine, synephrine and caffeine are usually added after the final portion of bulking agent and flavor have been added. The entire mixing procedure typically takes from five to fifteen minutes, but longer mixing times may sometimes be required. Those skilled in the art will recognize that many variations of the above described procedure may be followed.

The compositions may be taken orally by chewing. For oral administration the compositions may alternatively be formulated as a draught in water or in a syrup, in capsules, sachets, boluses mints or tablets, as an aqueous or oleaginous solution or suspension or in suspension in a syrup, such suspensions optionally including suspending agents, or as an oil-in-water or water-in-oil emulsion. Tablets may contain the active compounds as a powder or granules optionally mixed with binders, lubricants, inert diluents or surface-active or dispersing agents and may be formed by compression or by molding in inert liquid diluent. Such tablets may be scored and/or coated. Capsules and sachets may contain the active compounds alone as admixture or in a mixture with one or more other ingredients. Capsules may also contain the active compounds in aqueous or oleaginous solution suspension or emulsion optionally in association with other ingredients. Chewing gum formulations are preferred.

All the above formulations may be produced by standard processes comprising bringing the active compounds into association with one or more pharmaceutically acceptable carriers.

A unit dose of the composition of the present invention, preferably contains ephedrine in an amount of about 2.0 to 10 mg per dose, more usually about 4.5 mg per dose (or if present as Ma Huang extract Ephedra sinica)—ephedrine alkaloids—in an amount of about 56 mg); synephrine in an amount of about 2.0 to about 5 mg per dose, more usually about 3 mg per dose (or if present as bitter orange (Citrus aurantium in an amount of about 50 mg); and caffeine (typically anhydrous caffeine) in an amount of about 20-75 mg per dose, more usually about 50 mg per dose. A particularly preferred unit dose is about 4.5 mg ephedrine, 3 mg synephrine and 50 mg caffeine.

Such unit dose compositions may be taken by chewing two pieces (typically each about 2.3 g) for approximately 10 minutes prior to exercise. or during exercise.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. A chewing gum composition comprising synephrine, ephedrine and caffeine.

2. A composition according to claim 1, in unit dosage form.

3. A composition according to claim 2, wherein the synephrine is present in an amount of about 0.5 mg to 100 mg per dose.

4. A composition according to claim 3, wherein the synephrine is present in an amount of about 3 mg per dose.

5. A composition according to claim 2, wherein the ephedrine is present in an amount of about 0.5 mg to 100 mg per dose.

6. A composition according to claim 5, wherein the ephedrine is present in an amount of about 3-5 mg per dose.

7. A composition according to claim 1, wherein the caffeine is present in an amount of about 0.5-100 mg per dose.

8. A composition according to claim 7, wherein the caffeine is present in an amount of about 40-60 mg per dose.

9. A composition according to claim 1, wherein the chewing gum includes sugar.

10. A composition according to claim 1, wherein the chewing gum is substantially sugar-free.

11. A composition according to claim 1, wherein the chewing gum is low-calorie chewing gum.

12. A composition according to claim 1, wherein the chewing gum further comprises white willow.

13. A composition according to claim 12, wherein the white willow is present in an amount of about 0.5 mg to 3 grams.

14. A composition according to claim 1, wherein the chewing gum further comprises Schizandra chinensis in an amount of about 0.5 mg to 2 grams.

15. A composition according to claim 1, wherein the chewing gum further comprises iso-quescetin in an amount of about 0.5 mg to 1 gram.

16. A composition according to claim 1, wherein the chewing gum further comprises Quercetin in an amount of about 0.5 mg to 1 g.

17. A composition according to claim 1, wherein the chewing gum further comprises an additive selected from acesulfame-K, aspartame, saccharin, xylitol, monoammonium glycerrhizinate, glucono delta lactone, ethylmaltol, vanillin, creams, fruit flavors, and spices.

18. A method of oral delivery of synephrine, ephedrine and caffeine to a host, comprising administering a chewing gum composition comprising synephrine, ephedrine and caffeine.
